(19) United States
(12) Patent Application Publication
(10) Pub. No.: US 2005/0278124 A1
(43) Pub. Date: Dec. 15, 2005

(54) Methods for molecular property modeling using virtual data

(76) Inventors: Nigel P. Duffy, San Francisco, CA (US); Guido Lanza, San Francisco, CA (US); Jessen Yu, San Francisco, CA (US); William Mydlowec, San Francisco, CA (US)

Correspondence Address: RAYMOND R. MOSER JR., ESQ.;MOSER IP LAW GROUP, 1040 BROAD STREET, 2ND FLOOR, SHREWSBURY, NJ, 07702, US

(21) Appl. No.: 11/074,587

(22) Filed: Mar. 8, 2005

Related U.S. Application Data
(60) Provisional application No. 60/579,619, filed on Jun. 14, 2004.

(51) Int. Cl. G06F 19/00; G01N 33/48; G01N 33/50

(52) U.S. Cl. 702/19; 702/27

(57) ABSTRACT

Embodiments of the invention provide methods, systems, and articles of manufacture for modeling molecular properties based on information obtained from sources other than direct empirical measurements of the properties. Embodiments of the invention use “virtual data” related to molecular properties to train a molecular properties model. Virtual data about a molecule may include real-valued data (e.g. measurement values falling along a continuous range) or a positive or negative assertion about whether a molecule exhibits a property of interest. Virtual data may be generated using a variety of techniques and may be further characterized by confidence in the accuracy of the virtual data. In addition to virtual data, embodiments of the invention may use “virtual molecules” paired with “virtual data” to train a molecular properties model. The virtual molecules may themselves be generated in a variety of ways.

### CROSS REFERENCE TO RELATED APPLICATIONS

This application claims priority to U.S. Provisional Application Ser. No. 60/579,619, filed on Jun. 14, 2004, incorporated by reference herein in its entirety. This application is related to commonly owned U.S. Pat. No. 6,571,226 entitled “Method and Apparatus for Automated Design of Chemical Synthesis Routes,” which is incorporated by reference herein in its entirety.

### BACKGROUND OF THE INVENTION

1. Field of the Invention

The present invention relates to machine learning. More particularly, the present invention relates to methods, systems and articles of manufacture for constructing a molecular properties model that includes using virtual molecules and virtual data.

2. Description of the Related Art

Many industries use machine learning techniques to construct models of relevant phenomena. For example, machine learning applications have been developed that detect fraudulent credit card transactions, predict creditworthiness, or recognize words spoken by an individual. More generally, machine learning techniques may be used to construct software applications that improve their ability to perform a task with experience. Often, the task is to predict an unknown attribute or quantity from known information (e.g., credit risk predictions based on prior lending history), or to classify an object as belonging to a particular group (e.g., speech recognition software that classifies speech into individual words). Typically, a machine learning application gains experience using a set of training examples. The training examples may include both a description of the known information or object to be classified, along with a value for the otherwise unknown attribute or the correct classification of the object. For example, speech recognition software may be trained by having a user recite a pre-selected paragraph of text.

In bioinformatics and computational chemistry, machine learning applications may be used to develop a model of a molecular property. Such a model is configured to predict whether a particular molecule will exhibit the property being modeled. For example, models may be developed that predict biological properties such as pharmacokinetic, pharmacodynamic properties, physiological or pharmacological activity, toxicity or selectivity. Models may also be developed that predict chemical properties such as reactivity, binding affinity, or properties of specific atoms or bonds in a molecule, e.g. bond stability. Similarly, models may be developed that predict physical properties such as melting point or solubility. Models may also be developed that predict properties useful in physics based simulations such as force-field parameters.

The training examples used to train a molecular properties model typically include descriptions for a set of molecules (e.g., the atoms in a particular molecule along with the bonds between them) and data regarding the property of interest for each molecule included in the set. Collectively, the training examples are commonly referred to as a “training set” or as “training data.” The training data may be obtained from empirical measurements of the property of interest for a set of known molecules, or from published results thereof. Once the training examples are used to train the model, molecule descriptions representing additional molecules may be applied to the input of the trained model, which then outputs predictions regarding the property of interest for the additional molecules.

Often, the training data will include a disproportionate number of molecules known to exhibit the molecular property being modeled. For example, scientific articles often report only molecules that have a particular property of interest, and not those determined not to have the property of interest. Training a model using only this “positive data,” however, may bias the resulting model such that it will generate inaccurate predictions. One solution to this is to include molecules in the training set that are known to not have the property of interest. Problems arise, however, because molecules lacking the property of interest may not be known, or at least, have not been reported. Additionally, there may only be a very limited number of molecules known to have (or not to have) the property of interest at all. In some cases, therefore, there is an insufficient amount of data related to the property of interest available to train a molecular properties model, or there is an insufficient ratio between molecules known to have the property of interest and those known to not have the property of interest. Furthermore, for many properties of interest, there may simply not be data available for any molecules at all.

In these cases, generating the required data from laboratory experimentation may be both costly and time consuming. Moreover, a significant motivation for using machine learning techniques to generate a model of a molecular property is to avoid the very expense of performing laboratory experimentation. Accordingly, there remains a need for improved techniques for modeling molecular properties, and in particular, for generating a set of training data used to train a molecular properties model.

### SUMMARY OF THE INVENTION

Embodiments of the invention provide methods for modeling molecular properties based on information obtained from sources other than direct empirical measurements of the properties. Embodiments of the invention use “virtual data” related to molecular properties to train a molecular properties model. Virtual data about a molecule may include, for example, real-valued data (e.g., measurement values within a continuous range), a positive or negative assertion about whether a molecule exhibits a property of interest or an assertion regarding the ordering, or relative magnitude, of two or more molecules relative to the property of interest.

In some embodiments, virtual data may be generated using a variety of methods including random assignment, predictions from other predictive methods such as docking, and the like. As those skilled in the art will recognize, docking is a computational simulation technique where a molecule is assigned a predicted activity based on the compatibility of its 3-dimensional structure with the 3-dimensional structure of a protein. A particular example of docking is using molecular mechanics simulations to predict the free energy of binding.

Virtual data may be further characterized by a measure of confidence in the accuracy of the virtual data. (e.g., by random guess, estimated prior percentages, human expert labeled). In addition, embodiments of the invention may use “virtual molecules” along with “virtual data” to train a molecular properties model. The virtual molecules may themselves be generated in a variety of ways (e.g., by virtual synthesis). Embodiments of the invention further provide methods for generating training data used to train a molecular properties model. In one embodiment, the method generally includes selecting a set of molecules, wherein each member of the set of molecules is selected from (i) molecules known to have, or to not have, a property of interest, (ii) molecules presumed to have, or to not have, the property of interest, (iii) virtual molecules, wherein each virtual molecule is presumed to have, or to not have, the property of interest, and wherein the set of molecules is used to train a molecular properties model.

The method also includes, generating a representation of the molecules included in the set of molecules in a form appropriate for a selected machine learning algorithm, providing the representation of the molecules to the selected machine learning algorithm, and outputting a learned molecular properties model. Generally, the machine learning algorithm processes the representations of the molecules to generate a molecular properties model. The learned molecular properties model may then be used to generate a prediction about the property of interest for additional molecules. Additional molecules predicted to exhibit the property of interest may then be the subject of further investigation, e.g., experimental verification of the prediction.

### BRIEF DESCRIPTION OF THE DRAWINGS

The following detailed description makes reference to the drawings, which are now briefly described.

FIG. 1 illustrates an exemplary computer system that may be used to implement or perform embodiments of the present invention.

FIG. 2 is a block diagram illustrating sources of training data, including data sources used to provide virtual data and virtual molecules used to train a molecular properties model, according to one embodiment of the invention.

FIG. 3 illustrates a flow diagram of a method for constructing a molecular properties model using virtual data, according to one embodiment of the invention.

FIG. 4 illustrates a block diagram of data flow using a molecular properties model to generate predictions for arbitrary molecules, according to one embodiment of the invention.

### DETAILED DESCRIPTION

Embodiments of the present invention provide methods and articles of manufacture for generating training data used to train a molecular properties model (“model” for short). Embodiments of the invention provide training data that includes descriptions of molecules known to physically exist along with descriptions of molecules generated in silico using computational means, i.e., “virtual molecules.” Virtual molecules may be constructed using computational simulations that generate molecules capable of physically existing, but which may never have been physically synthesized. As used herein, property information or “property of interest” generally refers to a molecular property being modeled.

In one embodiment, the property information represents an empirically measurable property of a molecule. The property information for a given molecule may be based on intrinsic or extrinsic properties including, for example, the physiological activity, pharmacokinetic property, pharmacodynamic property, physiological or pharmacological activity, toxicity or selectivity; a chemical property including reactivity, binding affinity, or a property of specific atoms or bonds in a molecule; or a physical property including melting point or solubility or a force-field parameter.

Typically, the task of the model is to generate a prediction about the property of interest relative to a particular test molecule (whether the test molecule is selected from real, existing, known or virtual molecules). The model learns to perform the task using training data provided by embodiments of the invention. Further, property information for molecules included in the training data may be provided using “virtual data,” and may include information obtained from reasonable assumptions, computer simulations, or other modeling efforts. For example, computer simulations may be performed that simulate the physics of the molecular property of interest using molecular mechanics or quantum mechanics. Property information may also be obtained from laboratory experimentation or published literature sources. Additionally, property information may include a measure of “confidence” or belief in the validity or accuracy of the property information for a particular molecule.

Although this description refers to embodiments of the invention, the invention is not limited to any specifically described embodiments; rather, any combination of the described features, whether related to a described embodiment or not, implements the invention. Further, although various embodiments of the invention may provide advantages over the prior art, whether a given embodiment achieves a particular advantage, does not limit the invention. Thus, the features, embodiments, and advantages described herein are illustrative and should not be considered elements or limitations, except those explicitly recited in a claim. Similarly, references to “the invention” should neither be construed as a generalization of the inventive subject matter disclosed herein nor considered an element or limitation of the invention, unless explicitly recited in a claim.

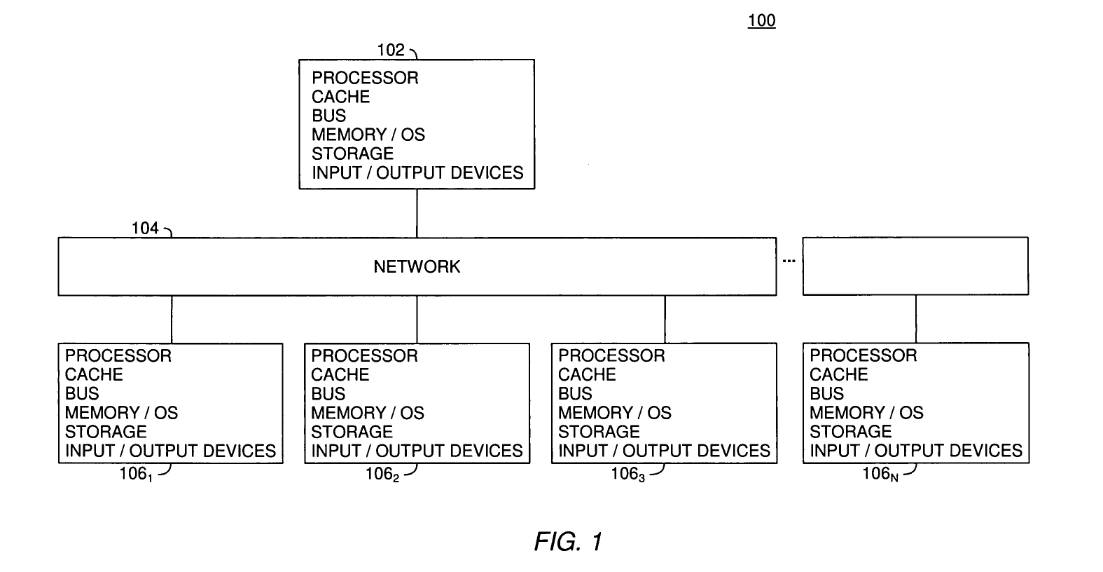

FIG. 1 illustrates a networked computer system 100 that may be used to implement or perform embodiments of the invention. Note however, that FIG. 1 illustrates only a particular embodiment of a networked computer system, and other embodiments are contemplated. Network 104 is used to connect computer system 102 and computer systems 106. In one embodiment, computer system 102 comprises a server configured to respond to the requests of systems 106. Computer systems 102 and 106 generally include a central processing unit (CPU) connected via a bus to memory and storage devices. Typical storage devices include IDE, SCSI, or RAID managed hard drives, and memory devices include SDRAM and DDR memory modules.

Computer systems 106 and 102 are each running an operating system (e.g., a Linux® distribution, Microsoft Windows®, IBM's AIX®, FreeBSD, etc.) responsible for the control and management of hardware, and for basic system operations, as well as running software applications. Computer systems 106 and 102 may also include I/O devices such as a mouse, keyboard, display device, and other specialized hardware. Additionally, although FIG. 1 illustrates a client/server architecture, embodiments of the invention may be implemented in a single computer system, or in other configurations, such as peer-to-peer or distributed architectures. Further, the computer systems used to practice the methods of the present invention may be geographically dispersed across local or national boundaries using network 104. Moreover, predictions generated for a test molecule at one location may be transported to other locations using well known data storage and transmission techniques, and predictions may be verified experimentally at the other locations. For example, a computer system may be located in one country and configured to generate predictions about the property of interest for a selected group of molecules, this data may be then be transported (or transmitted) to another location, or even another country, where it may be the subject of further investigation e.g., laboratory confirmation of the prediction or further computer-based simulations.

In one embodiment, network 104 connects computer systems 102 and 106 to form a high-speed computing cluster, such as a Beowulf cluster, or other parallel configuration. Those skilled in the art will recognize that a computing cluster provides a high-performance parallel computing environment constructed from commonly available personal computer hardware. In such an embodiment, computer system 102 may comprise a master computer used to control and direct the scheduling and processing activity of computer systems 106.

As described above, a molecular properties model may be configured to generate predictions regarding a property of interest for a molecule supplied to the model as input data. In one embodiment, the model is constructed using machine learning techniques. Machine learning techniques use descriptions of molecules together with property information regarding the property of interest to generate a trained model. Different models may be configured to predict whether a test molecule is “active” or “inactive” (i.e., it predicts presence or absence of the property of interest); to predict an activity value from a range; or to predict the ranking of a test molecule as more or less active than another test molecule.

One choice faced in constructing a molecular properties model is the selection of the molecules and property information used to train the model. Once selected, a software application configured to perform a machine learning algorithm uses the training data to generate a molecular properties model. In one embodiment, training data may be represented using a set of ordered tuples like the ones listed below:
<molecule1, positive>
<molecule2, positive>
<molecule3, negative>
   In this representation, molecule1 and molecule2 are known to be positive for the property of interest. Accordingly, the property information for these molecules indicates “positive,” signifying that molecule1 and molecule2 exhibit the property of interest. In addition, “negative data” may also be used to train the model. For example, in the above representation, molecule3 is known to be negative for the property of interest. Accordingly, the property information for this molecule indicates “negative,” signifying that molecule3 does not exhibit the property of interest. A model trained using these training examples may be configured to predict whether additional molecules are positive or negative for the property of interest.

As described above, however, there is often an insufficient amount of data available to train a model. This may occur when there is inadequate availability of property information, relative to specific molecules, available to train a model. Embodiments of the invention provide for selecting training data (i.e., molecules) from novel sources. In addition to using known molecules with available data regarding a property of interest, embodiments of the invention may train a model using “virtual molecules” and “virtual data.” Embodiments of the invention select molecules to include in the training data for which a value for the property of interest are assigned using virtual data. Also, embodiments of the invention may include virtually generated molecules in the training data. Virtual data may include data based on reasonable assumptions about a randomly selected molecule or a virtually generated molecule. Additionally, combinations of virtual data and virtual molecules may be used. Together, virtual molecules and virtual data greatly expand the available pool of molecules that may be selected for inclusion in a set of training data.

Often, the assumed, or virtually generated, property information for these molecules will indicate that the randomly selected or virtually generated molecule is negative for a property of interest, or that they have a low activity value for a property of interest. This is effective because, oftentimes, only a very small percentage of molecules will exhibit a particular property of interest. Thus, the assumption that a particular molecule will be negative for a property of interest will typically prove to be correct. In addition to providing property information using reasonable assumptions, property information for a known molecule (or for a virtual molecule) may be provided using virtual data generated using computer simulations.

Sometimes, the property of interest may be overwhelmingly likely to occur. In such a case, only a limited number of molecules may be known for which the property is known to be negative. For example, some ion channels on the surface of a cell or cellular structure (e.g., an organelle) may be fairly porous, permeable by most of the molecules typically present in the channel's normal environment. In such cases, randomly selected molecules may include virtual data indicating that the molecule (or virtual molecule) is positive for the property of interest (or has a high activity score).

Including property information based on reasonable assumptions, or based on virtual data, may sometimes lead to inaccurate property information for some of the training examples included in the training data. Many learning algorithms, however, are resistant to such noise. That is, including some training examples with incorrect or inaccurate property information will not lead to a poorly performing model. Thus, including a small number of molecules in the training data with incorrect property information is acceptable.

In one embodiment, molecules may be obtained by randomly selecting molecules from a database of known molecules. In addition, selection criteria may be applied to limit the selection. Examples of selection criteria may include molecular weight, solubility, presence (or absence) of certain substituent groups, and the like. The selection criteria may be used to increase the accuracy of virtual data generated from assumed property information for randomly selected molecules (whether virtual or real).

Additionally, virtual molecules may be included in the training data. Virtual molecules may be generated using a variety of methods. In one embodiment, virtual molecules are generated using the techniques disclosed in commonly owned U.S. Pat. No. 6,571,226, entitled, “Method and Apparatus for Automated Design of Chemical Synthesis Routes.” The '226 patent discloses methods of generating synthesizable virtual molecules using known reaction pathways and starting molecules, even though the “generation” is carried out using a computer-based simulation, and not laboratory synthesis practices. Doing so generates virtual molecules that are both physically realizable (i.e., molecules that conform to physical laws), and that may be actually synthesized (i.e., obtained in useful quantities) using known reaction pathways, and that may further satisfy goals or criteria in the synthesis route. The techniques disclosed in the '226 patent may be used to generate a set of virtual molecules included in the training data used to train a molecular properties model. Other methods of generating virtual molecules, however, may be used.

In one embodiment, other known properties of a molecule may be used to decide whether to include (or exclude) a particular molecule in a training set. For example, the solubility of a particular molecule may be unrelated to the property of interest, even though all the known molecules that exhibit the property of interest turn out to be soluble. In this case, molecules (or virtual molecules) may be filtered based on solubility. Molecules identified as soluble are then assumed to be negative for the property of interest and included in the training data. Including a set of soluble, yet assumed negative, molecules in the training data prevents the model from identifying solubility as a property linked to the property of interest during the model construction.

In addition to using virtual data and virtual molecules to generate a set of training data, the training examples may be labeled with an indication of confidence about the accuracy of the property information for the training example. For example, if 80% of the known molecules with a particular substituent group are known to be positive for the property of interest, molecules in the training data with the substituent group are labeled with a greater probability of having the property of interest than a randomly selected molecule.

Further, labeling training examples with a measure of confidence allows specific molecules to be included more than once in the training data. For example, a given set of training data might include labeling a molecule as being positive with a confidence value of 95% for a first training example and also as being negative with a confidence value of 5% in a second training example. Labeling a training example with both positive and negative probabilities allows the model to use the same molecule more than once during the training process to reflect different possibilities about the molecule and the property of interest, based on the probability of each possibility.

Training a Molecular Properties Model

Using any, or all, of the above described techniques, a set of training data used to train a molecular properties model is selected. The training data may include training examples based on virtual molecules. Virtual data may be used to provide property information for both known molecules and virtual molecules.

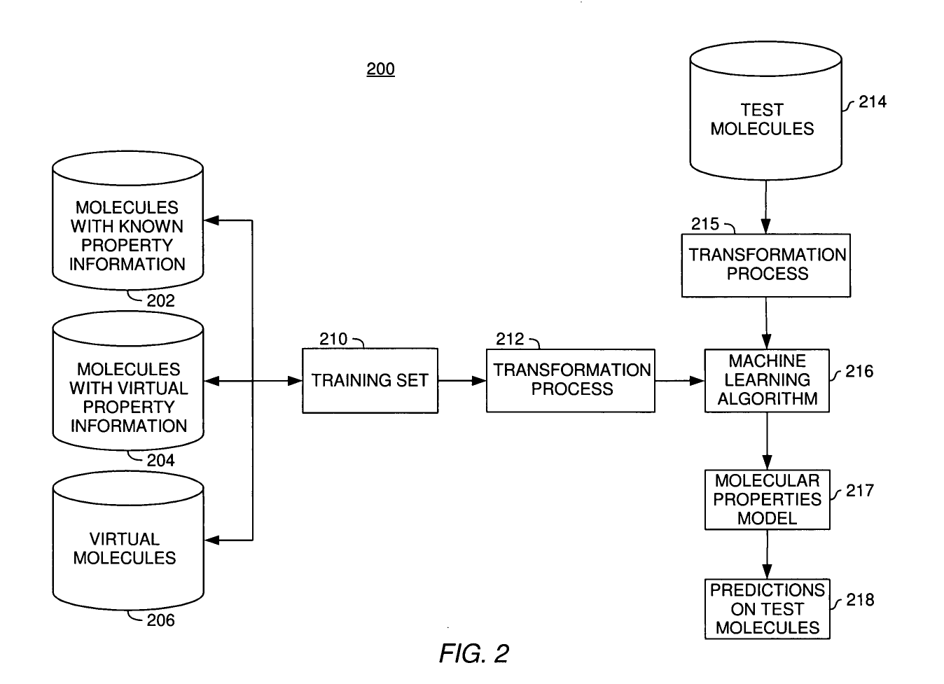

FIG. 2 illustrates data sources used to select molecules to include in the training data, according to one embodiment of the invention. Data sources 202-206 illustrate the different data sources described above. Data source 202 illustrates a database of known molecules. Molecules selected from data source 202 are both known to exist and have property information for the property of interest obtained through laboratory experimentation. Data source 204 illustrates known molecules for which property information for the property of interest is unavailable. Property information for these molecules may be provided using, for example, the techniques described above (e.g., using reasonable assumptions or generated using computational simulations).

Data source 206 represents virtual molecules that may be included in the training set. The property information for a training example that includes a virtual molecule may be generated using, for example, any of the techniques described above (e.g., assumption, in silico simulation of properties, and the like). In one embodiment, a set of molecules selected from data sources 202-206 are combined to form a plurality of training examples. Each training example includes a representation of the molecule and also includes property information for the molecule. Additionally, for molecules selected from data sources 202-206, the training example may further include a measure of confidence in the accuracy of the property information. In one embodiment, virtual molecules, or virtual data about known molecules may be used to provide a training set with a roughly equal amount of positive and negative training examples. Once the set of training data is selected, transformation process 212 generates a representation of the molecules appropriate for a selected machine learning algorithm.

In one embodiment, the transformation process 212 may include creating a vector representation of the molecule included in a training example, or performing a conformational analysis of the molecule. Generally, as those skilled in the art will recognize, molecule representations are configured to encode the structure, features, and properties of the molecule that may account for its physical properties. Accordingly, features such as functional groups, steric features, electron density and distribution across a functional group or across the molecule, atoms, bonds, locations of bonds, and other chemical or physical properties of the molecule may be encoded by the representation of a molecule generated by transformation process 212.

Once the training examples are in an appropriate form, they may be provided to a software application 216 that is configured to execute a machine learning algorithm. The software application 216 takes the training examples as input for the selected machine learning algorithm. The software application 216 then constructs molecular properties model 217, according to the learning algorithm.

Subsequently, molecules selected from data source 214 may be provided to the model 217. Molecules selected from data source 214 may include additional molecules selected from sources 202-206, and processed for the model using transformation process 215. The transformation process 215 generates a representation of a test molecule appropriate for the particular model 217. The model 217 then generates a prediction about the property of interest for each such molecule. Molecules predicted to exhibit the property of interest may subsequently be the subject of further investigation, including experimentation carried out in the laboratory, or using computer simulation techniques.

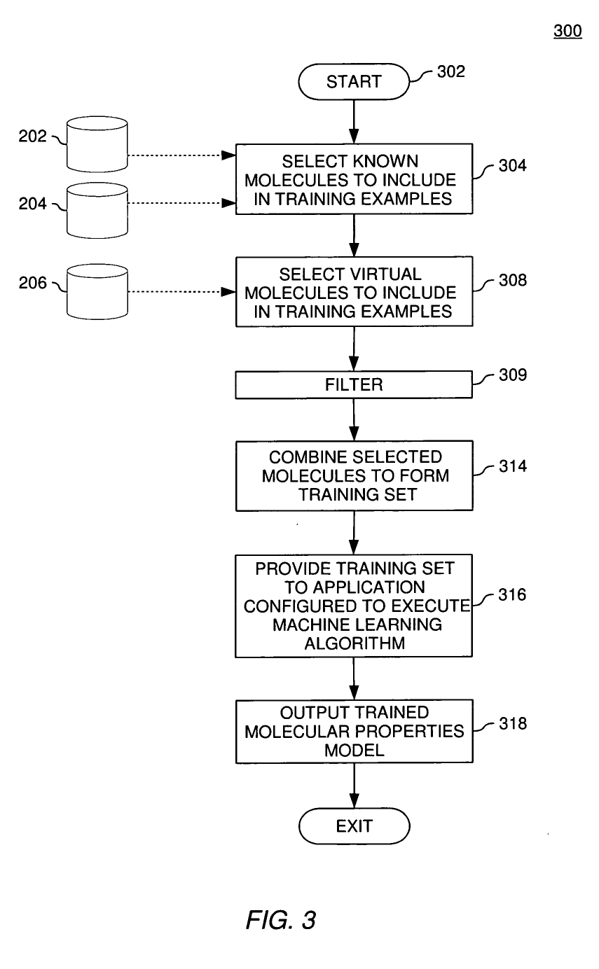

FIG. 3 depicts a flow diagram of a method that may be used to construct a molecular properties model, according to one embodiment of the invention. The method 300 begins at step 302 and proceeds to step 304. At step 304, molecules are selected to be included in the training data. For example, known molecules with known property information are selected from data source 202, and known molecules with property information generated using virtual data are selected from data source 204. At step 308, virtual molecules are selected from data source 206. Optionally, at step 309, the molecules selected from data sources 202, 204 and 206 are filtered based on characteristics such as similarity to molecules known to exhibit (or to not exhibit) the property of interest, or based on the presence (or absence) of other properties.

In step 314, molecules selected from data sources 202, 204, and 206 are combined to produce a set of training examples. In one embodiment, molecules in the training set are labeled with a measure of confidence regarding the accuracy of the property information.

Next, at step 316, the set is provided to a software application configured to perform a machine learning algorithm (e.g., software application 216). At step 316 an arbitrary machine learning algorithm may learn from the training examples included in the training data. Various embodiments may use learning algorithms such as Boosting, a variant of Boosting, Alternating Decision Trees, Support Vector Machines, the Perceptron algorithm, Winnow, the Hedge Algorithm, an algorithm constructing a linear combination of features or data points, Decision Trees, Neural Networks, Genetic Algorithms, Genetic Programming, logistic regression, Bayes nets, log linear models, Perceptron-like algorithms, Gaussian processes, Bayesian techniques, probabilistic modeling techniques, regression trees, ranking algorithms, Kernel Methods, Margin based algorithms, or linear, quadratic, convex, conic or semi-definite programming techniques or any modifications of the foregoing, to learn from the training data selected during step 314. Further, embodiments of the present invention contemplate using machine learning algorithms developed in the future, including newly developed algorithms or modifications of the above listed learning algorithms.

Once learning is complete, a molecular properties model is output at step 318. The molecular properties model output at step 318 is configured to generate a prediction regarding the property of interest for an arbitrary molecule supplied as input to the model.

The Trained Molecular Properties Model

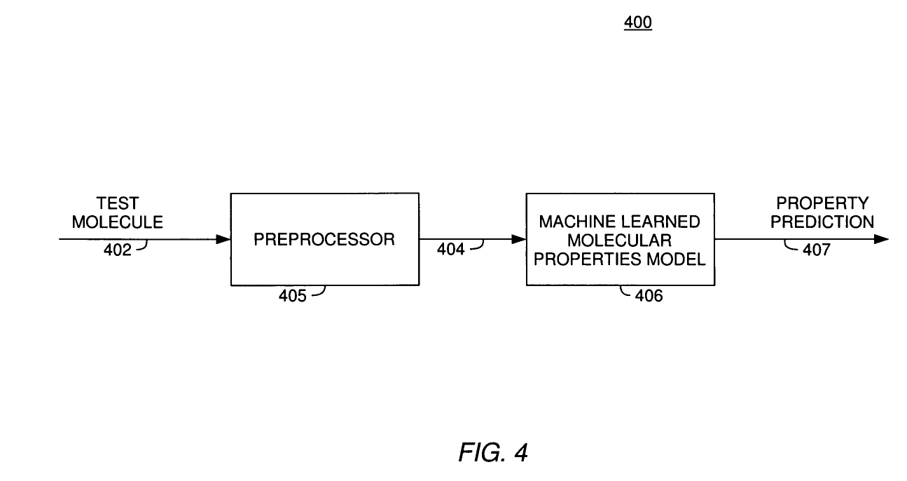

FIG. 4 illustrates a block diagram of a data flow 400 for using the trained molecular properties model to generate predictions regarding arbitrary molecules, according to one embodiment of the invention. The data flow 400 includes a molecule description preprocessor 405 and learned model 406 (e.g., the model output at step 318 of the method illustrated in FIG. 3).

Model 406 may be configured to predict whether an arbitrary test molecule will exhibit the property of interest. Molecule descriptions are applied to path 402. In one embodiment, the molecule descriptions may be generated using the same techniques used for the training examples. The preprocessor 405 processes descriptions of the test molecules to create suitable inputs for the model 406. That is, test molecules may be transformed into a representation according to the transformation process 212 described above in reference to FIG. 2. Once supplied to the model 406 on input path 404, the model 406 generates a prediction about the test molecule by applying the model to the test molecule. The model 406 outputs the prediction on output path 407.

While the foregoing is directed to embodiments of the present invention, other and further embodiments of the invention may be devised without departing from the basic scope thereof.

1. A method for generating a set of training data used to train a molecular properties model, comprising:
selecting virtual molecules, wherein the virtual molecules are generated using a software application configured to generate representations of physically possible molecules;
assigning the virtual molecules a value for a property of interest being modeled, wherein the property of interest comprises an empirically measurable property, and wherein at least one virtual molecule is assigned an assumed value for the property of interest; and
forming the set of training data from the selected virtual molecules and assigned values for the property of interest.

2. The method of claim 1, wherein the value assigned to a given molecule included in the set of training data comprises an indication that the given molecule is “active” or “inactive” for the property of interest, a prediction of the activity of the given molecule selected from within a continuous range of values, a prediction that the given molecule is more or less active than another molecule, or a prediction regarding the relative magnitude or differences in the property of interest for two or more molecules included in the set of training data.

3. The method of claim 1, wherein the empirically measurable property comprises a physiological activity, pharmacokinetic property, pharmacodynamic property, physiological or pharmacological activity, toxicity or selectivity; a chemical property including reactivity, binding affinity, or a property of a specific atom or bond in a molecule; or a physical property including melting point, solubility, a membrane permeability, or a force-field parameter.

4. The method of claim 1, wherein at least one virtual molecule is generated by selecting a product of a simulation of a chemical reaction pathway or of a plausible chemical reaction simulated by the software application.

5. The method of claim 1, wherein assigning the value to at least one molecule included in the set of training data comprises, running a computer simulation configured to simulate plausible chemical or physical processes involving the at least one molecule or to simulate properties of the at least one molecule.

6. The method of claim 1, wherein assigning the value to at least one molecule included in the set of training data comprises, assigning the most statistically likely value of the property for interest for a randomly selected molecule.

7. The method of claim 1, further comprising:
generating a representation of the molecules included in the set of training data in a form appropriate for a second software application, wherein the second software application is configured to perform a machine learning algorithm using the set of training data; and
providing the set of training data to the second software application,
performing the machine learning algorithm, thereby generating the molecular properties model.

8. The method of claim 7, wherein generating a representation of the molecules included in the set of training data further comprises, including a confidence value for a molecule in the set of training data, wherein the confidence value indicates a measure of confidence in the accuracy of the assigned value relative to the true value for the property of interest and the molecule.

9. The method of claim 7, further comprising:
selecting a test molecule;
generating a representation of the test molecule appropriate for the molecular properties model; and
providing the representation of the test molecule to the molecular properties model; and
generating a prediction about the property of interest for the test molecule.

10. The method of claim 9, further comprising, determining the accuracy of the prediction for the test molecule by carrying out laboratory experimentation using physically existing samples of the test molecule.

11. The method of claim 9, further comprising, determining the accuracy of the prediction for the test molecule by performing a research study using physical samples of the test molecule.

12. A method of generating training data used to train a molecular properties model, the method comprising:
selecting virtual molecules, wherein the virtual molecules are generated using a software application configured to generate representations of physically possible molecules;
assigning the virtual molecules a value for a property of interest being modeled, wherein the property of interest comprises an empirically measurable property, and wherein at least one virtual molecule is assigned an assumed value for the property of interest; and
forming the set of training data from the selected virtual molecules and assigned values for the property of interest;
generating a representation of the molecules included in the set of training data in a form appropriate for a second software application, wherein the second software application is configured to perform a machine learning algorithm using the set of training data; and
providing the set of training data to the second software application, performing the machine learning algorithm, thereby generating the molecular properties model;
selecting a test molecule;
generating a representation of the test molecule appropriate for the molecular properties model; and
providing the representation of the test molecule to the molecular properties model; and
generating a prediction about the property of interest for the test molecule.

13. A method for generating a set of training data used to train a molecular properties model, comprising:
selecting molecules;
assigning the molecules a value for the property of interest being modeled, wherein the property of interest comprises an empirically measurable property, and wherein at least one molecule is assigned an assumed value for the property of interest;
forming the set of training data from the selected molecules and assigned values for the property of interest.

14. The method of claim 13, wherein the value assigned to a given molecule included in the set of training data comprises an indication that the given molecule is “active” or “inactive” for the property of interest, a prediction of the activity of the given molecule selected from within a continuous range of values, a prediction that the given molecule is more or less active than another molecule, or a prediction regarding the relative magnitude or differences in the property of interest for two or more molecules included in the set of training data.

15. The method of claim 13, wherein the empirically measurable property for the at least one molecule comprises a physiological activity, pharmacokinetic property, pharmacodynamic property, physiological or pharmacological activity, toxicity or selectivity.

16. The method of claim 13, wherein the empirically measurable property for the at least one molecule comprises a chemical property selected from at least one of reactivity, binding affinity, a property of a specific atom or a bond in a molecule.

17. The method of claim 13, wherein the empirically measurable property for the at least one molecule comprises a physical property selected from at least one of a solubility, a membrane permeability, or a force-field parameter.

18. The method of claim 13, wherein at least one virtual molecule is generated by selecting a product of a simulation of a chemical reaction pathway or of a plausible chemical reaction simulated by the software application.

19. The method of claim 13, wherein assigning the value to at least one molecule included in the set of training data comprises, assigning, to the at least one molecule, the most statistically likely value of the property for interest for a randomly selected molecule.

20. The method of claim 13, wherein assigning the value to at least one molecule included in the set of training data comprises, running a computer simulation configured to simulate plausible chemical or physical processes involving the at least one molecule or to simulate properties of the at least one molecule.

21. The method of claim 13, further comprising:
generating a representation of the molecules included in the set of training data in a form appropriate for a second software application, wherein the second software application is configured to perform a machine learning algorithm using the set of training data; and
providing the set of training data to the second software application, performing the machine learning algorithm, thereby generating the molecular properties model.

22. The method of claim 21, wherein generating a representation of the molecules included in the set of training data comprises, determining plausible three-dimensional conformations of the molecules based on the atoms and bonds between atoms present in a given molecule; or comprises, generating a vector representation of the molecules, wherein the vector representation is configured to encode the structure of a given molecule included in the set of training data.

23. The method of claim 21, wherein generating a representation of the molecules included in the set of training data further comprises: including a confidence value for a molecule in the set of training data, wherein the confidence value indicates a measure of confidence in the accuracy of the assigned value relative to the true value for the property of interest and the molecule.

24. The method of claim 21, wherein the learning algorithm is selected from one of Boosting, a variant of Boosting, Alternating Decision Trees, the Perceptron algorithm, Winnow, the Hedge Algorithm, an algorithm constructing a linear combination of features or data points, logistic regression, Bayes nets, log linear models, Perceptron-like algorithms, Gaussian processes, probabilistic modeling techniques, regression trees, ranking algorithms, margin based algorithms, or linear, quadratic, convex, conic or semi-definite programming techniques and any combinations thereof.

25. The method of claim 21, further comprising:
selecting a test molecule;
generating a representation of the test molecule appropriate for the molecular properties model; and
providing the representation of the test molecule to the molecular properties model; and
generating a prediction about the property of interest for the test molecule.

26. The method of claim 25, further comprising, determining the accuracy of the prediction for the test molecule by carrying out laboratory experimentation using physically existing samples of the test molecule.

27. The method of claim 25, further comprising, determining the accuracy of the prediction for the test molecule by performing a research study using physical samples of the test molecule.

28. A computer-readable medium containing an executable component that, when executed by a processor, performs operations comprising:
selecting virtual molecules, wherein the virtual molecules are generated using a software application configured to generate representations of physically possible molecules;
assigning the molecules a value for the property of interest being modeled, wherein the property of interest comprises an empirically measurable property, and, wherein at least one virtual molecule is assigned an assumed value for the property of interest; and
forming the set of training data from the selected virtual molecules and assigned values for the property of interest.

29. The computer-readable medium of claim 28, wherein the software application is configured to generate virtual molecules by selecting a product of a simulation of a chemical reaction pathway or of a plausible chemical reaction simulated by the software application.

30. The computer-readable medium of claim 28, wherein assigning the value to at least one molecule included in the set of training data comprises, running a computer simulation configured to simulate plausible chemical or physical processes involving the at least one molecule or to simulate properties of the at least one molecule.

31. The computer-readable medium of claim 28, wherein the operations further comprise:
generating a representation of the molecules included in the set of training data in a form appropriate for a second software application, wherein the second software application is configured to perform a machine learning algorithm using the set of training data; and
providing the set of training data to the second software application,
performing the machine learning algorithm, thereby generating the molecular properties model.

32. The computer-readable medium of claim 31, wherein generating a representation of the molecules included in the set of training data further comprises, including a confidence value for a molecule in the set of training data, wherein the confidence value indicates a measure of confidence in the accuracy of the assigned value relative to the true value for the property of interest and the molecule.

33. The computer-readable medium of claim 31, wherein the operations further comprise:
selecting a test molecule;
generating a representation of the test molecule appropriate for the molecular properties model; and
providing the representation of the test molecule to the molecular properties model; and
generating a prediction about the property of interest for the test molecule.

34. The computer-readable medium of claim 33, wherein the prediction generated for the test molecule is selected from at least one of,
(i) a prediction that the test molecule is “active” or “inactive” for the property of interest,
(ii) a prediction of the activity of the test molecule within a continuous range of values,
(iii) a prediction that the test molecule is more or less active than another test molecule, or
(iv) a prediction regarding the relative magnitude of the property of interest between two or more molecules.

35. A computer-readable medium containing an executable component that, when executed by a processor, performs operations comprising:
selecting molecules;
assigning the molecules a value for the property of interest being modeled, wherein the property of interest comprises an empirically measurable property, and wherein at least one molecule is assigned an assumed value for the property of interest;
forming the set of training data from the selected molecules and assigned values for the property of interest.

36. The computer-readable medium of claim 35, wherein assigning the value to at least one molecule included in the set of training data comprises: running a computer simulation configured to simulate plausible chemical or physical processes involving the at least one molecule or to simulate properties of the at least one molecule.

37. The computer-readable medium of claim 35, wherein the operations further comprise:
generating a representation of the molecules included in the set of training data in a form appropriate for a second software application, wherein the second software application is configured to perform a machine learning algorithm using the set of training data; and
providing the set of training data to the second software application, performing the machine learning algorithm, thereby generating the molecular properties model.

38. The computer-readable medium of claim 37, wherein the operations further comprise:
selecting a test molecule;
generating a representation of the test molecule appropriate for the molecular properties model; and
providing the representation of the test molecule to the molecular properties model; and
generating a prediction about the property of interest for the test molecule.

39. A method for evaluating a prediction about a molecule, generated by a molecular properties model, comprising:
receiving the prediction for a test molecule generated by the molecular properties model, wherein the molecular properties model is trained using a set of training data, and wherein the training data comprises:
molecules generated using a first software application configured to generate representations of physically possible molecules; and
a value for a property of interest assigned to each molecule, wherein at least one molecule is assigned an assumed value for the property of interest,
determining the accuracy of the prediction for the test molecule by carrying out experimentation using physically existing samples of the test molecule.
